(19) United States
(12) Patent Application Publication
(10) Pub. No.: US 2008/0014641 A1
(43) Pub. Date: Jan. 17, 2008

(54) CORRECTING KARL FISCHER WATER AND OIL ANALYSIS

(76) Inventors: A. Andrew Carey, Lenoir City, TN (US); Anthony J. Hayzen, Knoxville, TN (US)

Correspondence Address: LUEDEKA, NEELY & GRAHAM, P.C., P O BOX 1871, KNOXVILLE, TN, 37901, US

(21) Appl. No.: 11/457,671

(22) Filed: Jul. 14, 2006

(51) Int. Cl. G01N 33/18

(52) U.S. Cl. 436/42

(57) ABSTRACT

A method and apparatus for determining a concentration of water in an oil mixture using both the Karl Fischer test method and spectrochemical analysis. The invention allows for chemical species that masquerade as water in an oil mixture to be identified and accounted for in determining a more accurate water concentration measurement than by using the Karl Fischer test method alone.

### FIELD

This invention relates to the field of chemical analysis. More particularly, this invention relates to a method and apparatus for more accurately determining the concentration of water in an oil mixture.

### BACKGROUND

Problems associated with water contamination in oil systems are well known in a vast array of industries and applications. Water in oil (whether in a dissolved, emulsified, or free form) has the potential to damage equipment and significantly reduce efficiency in systems that depend on a certain degree of oil purity. Examples of such equipment and systems include, but are not limited to, the transportation industries (i.e., aircraft, ship going vessels, and terrestrial motorized vehicles), the metal production and refining industries, various industries dependant on reliable hydraulic power. and industries producing lubricants for various applications. In all of the examples mentioned (and many more not mentioned), the need for high purity oil is paramount.

Due to the need for high purity oil in so many varying industries and applications, a number of techniques have been developed to determine or otherwise monitor the concentration of water in a given oil sample. One of the most widely used and well-known techniques is the Karl Fischer test method. The Karl Fischer test method typically includes a chemical reaction in which water in a mixture is made to react with sulfur dioxide and iodine as shown in the balanced chemical reaction equation given below.

2H₂O+SO₂+I₂→H₂SO₄+2HI

Other chemical species may be used, but the most common species are given in the example above for illustration only. The iodine and sulfur dioxide are typically dissolved in what is commonly called the Karl Fischer reagent. The solvent pyridine has been traditionally used in Karl Fischer reagents, but other solvents with less toxic characteristics such as kerosene have been used more recently instead of pyridine. After preparation, the Karl Fischer reagent (typically including the solvent kerosene, dissolved iodine, and dissolved sulfur dioxide) is then added to an oil mixture sample. Water content of the oil mixture sample is determined based on how much Karl Fischer reagent (i.e., how much iodine) is used to bring the reaction of the entire oil mixture sample to substantial completion.

The Karl Fischer test method in its various forms has been used for decades and is still a fundamental tool in the determination of the amount of water in an oil mixture. However, as various processes and applications using oil mixtures increase in efficiency and improve in performance, a higher degree of certainty with regard to water impurity in oil mixtures becomes necessary. As tolerances for precise Karl Fischer test results become narrower, the sufficiency of using the Karl Fischer test method by itself becomes strained.

Though the general Karl Fischer test method is reliable to give an indication of how much water is in a given oil mixture sample, the method has its drawbacks. A number of sources including U.S. Pat. No. 6,361,670 to Cedergren (incorporated herein by reference) describe the well known problem of certain chemicals in a given oil sample simulating water, thereby causing artificially high water concentration measurements in Karl Fischer test results. The proposed solution by Cedergen is to control the oxidation of some of the interfering reaction products, thereby limiting their ability to produce false water concentration measurements during Karl Fischer testing. U.S. Pat. No. 5,246,863 to Dahms (incorporated herein by reference) also discusses artificially high Karl Fischer test results (described as “drift”) and describes an automatic titrating device to address the problem. U.S. Pat. No. 5,401,662 to Matschiner et al. (incorporated herein by reference) describes the use of an improved solvent for the Karl Fischer test method that minimizes water-simulating side reactions. Finally, U.S. Pat. No. 4,211,614 to Eppstein et al. (incorporated herein by reference) describes an apparatus and a method to compensate for the drift caused by water simulating side reactions during Karl Fischer testing wherein electrolysis is used.

Therefore, it is well known in the art that side reactions during Karl Fischer testing cause artificially high water concentration measurements. As demonstrated by the assortment of solutions to this problem already discussed herein, the approaches to solving this problem vary. However, virtually all of the solutions that have been developed to combat the problem of artificially high Karl Fischer test results involve the manipulation of the Karl Fischer testing procedure itself. The Karl Fischer test method in its various forms has its limits, however, and for certain modern applications, no level of tweaking the Karl Fischer test method alone will suffice for future low tolerance applications.

What is needed, therefore, is a test method and apparatus that uses and benefits from traditional Karl Fischer testing strategies, but also supplements such strategies with an accurate means to determine the amount of side reaction products causing false readings and account for such side reaction products.

### SUMMARY

The above and other needs are met by a method to determine a concentration of water in an oil mixture composed of oil, water, and at least one other chemical species. The steps include (1) using the Karl Fischer test method to determine a Karl Fischer test result, (2) determining a concentration of the at least one other chemical species using spectrochemical analysis, and (3) calculating an accurate concentration measurement of water in the oil mixture based on the Karl Fischer test result and the concentration of the at least one other chemical species. In a preferred embodiment, the at least one other chemical species includes Magnesium Sterate. In another preferred embodiment, the at least one other chemical species includes Zinc Dialkyl Dithiophosphate (ZDDP). In a related embodiment, the general method described above includes the additional step of performing a crackle test on the oil mixture.

Certain embodiments of the invention described herein also include an apparatus for performing the method described above. In a preferred embodiment, the apparatus includes a Karl Fischer testing assembly for performing the Karl Fischer test method on a first sample of an oil mixture to produce a Karl Fischer test result. The apparatus also includes a spectrochemical device such as a spectrometer, a spectroscope, a spectrograph or other similar device to determine the type and amount of undesirable side reactants present in a second sample of the oil mixture. The apparatus also includes a synthesizing means for comparing the Karl Fischer test result with the result from the spectrochemical device. Based on the comparison between the Karl Fischer test result and the result from the spectrochemical device, an accurate measurement for the concentration of water in the oil mixture is calculated.

### BRIEF DESCRIPTION OF THE DRAWINGS

Further advantages of the invention are apparent by reference to the detailed description in conjunction with the figures, wherein elements are not to scale so as to more clearly show the details, wherein like reference numbers indicate like elements throughout the several views, and wherein:

FIG. 1 depicts a stepwise diagram indicating a preferred embodiment of the progression of steps for determining an accurate concentration of water in an oil mixture;

FIG. 2 depicts a stepwise diagram indicating another preferred embodiment of the progression of steps for determining an accurate concentration of water in an oil mixture;

FIG. 3 depicts a preferred embodiment of an apparatus for determining an accurate concentration of water in an oil mixture.

### DETAILED DESCRIPTION

The Karl Fischer test method used to determine a concentration of water in an oil mixture is widely used because of its relative accuracy. However, one of the drawbacks of using the Karl Fischer test method is that certain reaction byproducts and other chemical species within the oil mixture tend to give false high readings of water content. In other words, certain reaction byproducts and other chemical species within the oil mixture effectively masquerade as water. Various embodiments of the invention described herein offer a method and apparatus using spectrochemical analysis in addition to the Karl Fischer test method. More specifically, spectrochemical analysis is used to identify certain masquerading chemical species and calculate a true water concentration of an oil mixture by subtracting the masquerading effects of certain reaction byproducts and other chemical species from Karl Fischer test results, thereby giving a more accurate overall water concentration test result.

Certain chemical classes have been identified by various sources as causing false water readings when the Karl Fischer test method is used to determine a concentration of water in an oil mixture. These general chemical classes include, but are not limited to, aldehydes and ketones, strong amines, ammonia, ferric salts, hydrazine derivatives, hydroxylamine salts, mercaptans, sodium methylate, sulfuric acid, thioacids, and thiourea. Tests were conducted on four additives/contaminants that fall under the chemical classes listed above. The four chemical species tested were magnesium sterate, ferric oxide, diphenyl amine, and zinc dialkyl dithiophosphate (ZDDP). Oil mixtures for magnesium sterate, ferric oxide, and diphenyl amine were tested with 0 ppm additive/contaminant, 500 ppm additive/contaminant, 1000 ppm additive/contaminant, and 2000 ppm additive/contaminant. Six runs per additive/contaminant were made at each of the four listed concentration value (i.e., 0 ppm, 500 ppm, 1000 ppm, and 2000 ppm). The test results suggested that ferric oxide and diphenyl amine had no substantial effect of masquerading as water in the oil mixture. However, the test results for magnesium sterate showed a substantially direct correlation between the concentration of additive/contaminant and the false high concentration measurement of water. The test results for magnesium sterate are shown in Table 1 below.

TABLE 1

| Magnesium (ppm) | Karl Fischer Test Result of Water (ppm) |
|---|---|
| 0 | 42 |
| 500 | 978 |
| 1000 | 1892 |
| 2000 | 3715 |

Based on the test results for magnesium sterate shown in Table 1, a direct correlation between the concentration of magnesium sterate and false high concentration measurements of water is established. More specifically, for approximately every 1000 ppm of magnesium sterate present in the oil mixture, a false high water concentration measurement of 1800 ppm emerges in Karl Fischer test results. An algorithm based on the relationship illustrated by Table 1 allows for false high water concentration measurements based on magnesium sterate to be factored out of a final water concentration measurement. By using spectrochemical analysis techniques. the identity and concentration of magnesium sterate may be calculated and reconciled with corresponding Karl Fischer test results to give a more accurate water concentration measurement.

For example, if a Karl Fischer test result from an oil mixture is 1000 ppm water and spectrochemical analysis reveals that 500 ppm of magnesium sterate is also present in the oil mixture, a calculation may be performed based on the relationship shown in Table 1 to demonstrate that only about 100 ppm of water is actually present in the oil mixture. The other 900 ppm of “water” from the Karl Fischer test result is actually magnesium sterate masquerading as water. The method described above using spectrochemical analysis for correcting false high water concentration measurements when using the Karl Fischer test method can also be used with other chemical species.

Another set of tests were performed on ZDDP to develop an algorithm based on the relationship between the mass percentage of ZDDP and Karl Fischer test results for water concentration (ppm) in an oil mixture. The ZDDP/oil mixtures were tested at values of 0%, 1%, 2%, and 5% ZDDP as shown in Table 2 below. The results of this test, like the results of the test with magnesium sterate, demonstrate a direct correlation between the percentage of ZDDP present in the oil mixture and the water concentration measurement using the Karl Fischer test method. The zinc content (mass %) or the phosphorus content (mass %) of ZDDP molecules can be used as the basis for a comparison with Karl Fischer test results for water concentration. Interestingly, the mass of zinc and the mass of phosphorus in a molecule of ZDDP is substantially the same, causing their relationship with Karl Fischer test results for water concentration to also be very similar.

The general chemical formula for ZDDP is ((RO)₂PS₂)₂Zn wherein the “R” represents a simple alkyl group such as methyl or butyl. When R=methyl, the phosphorus concentration is about 1790 ppm and the zinc concentration is about 1880 ppm. When R=butyl, however, the measured phosphorus concentration is about 1250 ppm and the zinc concentration is about 1320 ppm. When the zinc concentration from ZDDP ranges from about 1320 ppm to about 1880 ppm in an oil mixture, a false reading of about 290 ppm of excess water emerges in Karl Fischer test results for water concentration. Similarly, when the phosphorus concentration from ZDDP ranges from about 1250 ppm to about 1790 ppm in an oil mixture, a false reading of about 290 ppm of excess water emerges in Karl Fischer test results for water concentration. When the zinc concentration ranges from about 440 ppm (correlating to a phosphorus concentration of 430 ppm) to about 640 ppm (correlating to a phosphorus concentration of 620 ppm) in an oil mixture, a false reading of about 100 ppm of excess water emerges in Karl Fischer test results for water concentration.

Zinc concentrations from ZDDP may be measured and relied on independently of phosphorus concentrations for correcting Karl Fischer test results. Similarly, phosphorus concentrations from ZDDP may be measured and relied on independently of zinc concentrations for correcting Karl Fischer test results. In a preferred embodiment, the concentration of zinc and phosphorus may be averaged so that a Karl Fischer test measurement for water concentration may be corrected based on both zinc concentration and phosphorus concentration.

TABLE 2

| Percentage of | |
|---|---|
| ZDDP | Karl Fischer Test Result of Water (ppm) |
| 0 | 40 |
| 1 | 235 |
| 2 | 504 |
| 5 | 1447 |

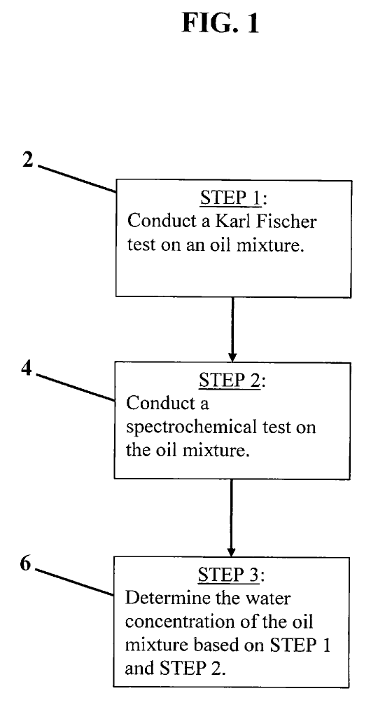

FIG. 1 depicts a preferred embodiment of a method for determining a concentration of water in an oil mixture composed of oil, water, and at least one other chemical species. In a first step 2, a Karl Fischer test is conducted on the oil mixture to obtain a Karl Fischer test result. The oil mixture is then tested as shown in a second step 4 using spectrochemical analysis techniques such as spectrometry, spectrography, spectroscopy, or other similar spectrochemical techniques to determine the identity and concentration of the one or more other chemical species. In a third step 6, an accurate concentration measurement of water in the oil mixture is calculated by subtracting or otherwise accounting for the masquerading effects of the at least one other chemical species. The first step 2 and the second step 4 need not be performed in any particular order. In fact, both the first step 2 and the second step 4 could be performed at substantially the same time.

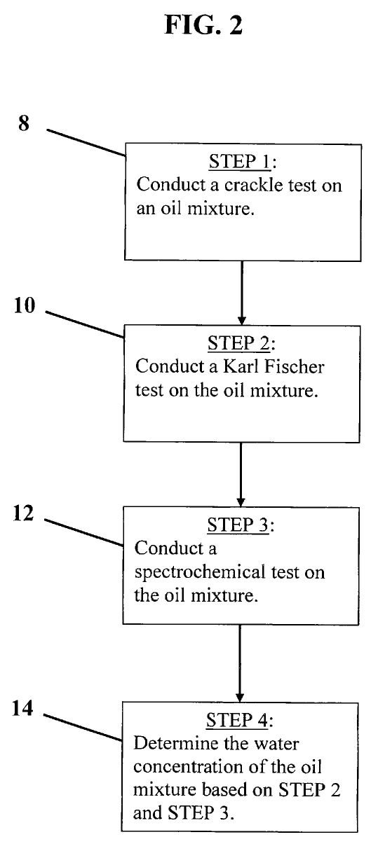

FIG. 2 illustrates a preferred embodiment including a first step 8 of conducting a crackle test to determine if suspended or free water is in an oil mixture. A second step 10 includes conducting a Karl Fischer test to produce a Karl Fischer test result. A third step 12 includes conducting a spectrochemical analysis of the oil mixture to identify and quantify the one or more other chemical species. A fourth step 14 includes calculating an accurate concentration measurement of water in the oil mixture by subtracting or otherwise accounting for the masquerading effects of the at least one other chemical species. As with the embodiment shown in FIG. 1, the first step 8, the second step 10, and the third step 12 do not have to occur in any particular order. All three steps (8, 10, and 12) could occur at substantially the same time.

The term “chemical species” used herein is meant to be interpreted broadly including one or more atoms of a pure element or a plurality of atoms of various elements bonded together by chemical bonding including, but not limited to, ionic bonding, covalent bonding, a combination of ionic bonding and covalent bonding, metallic bonding, and hydrogen bonding. The term “Karl Fischer test method” or “Karl Fischer test” is meant to be interpreted broadly to include all test methods known to those skilled in the art that are commonly referred to as or, otherwise, are understood by one of ordinary skill in the art to be Karl Fischer testing methods including, but not limited to, methods using solvents such as pyridine, benzene, and other Karl Fischer solvents known to those skilled in the art.

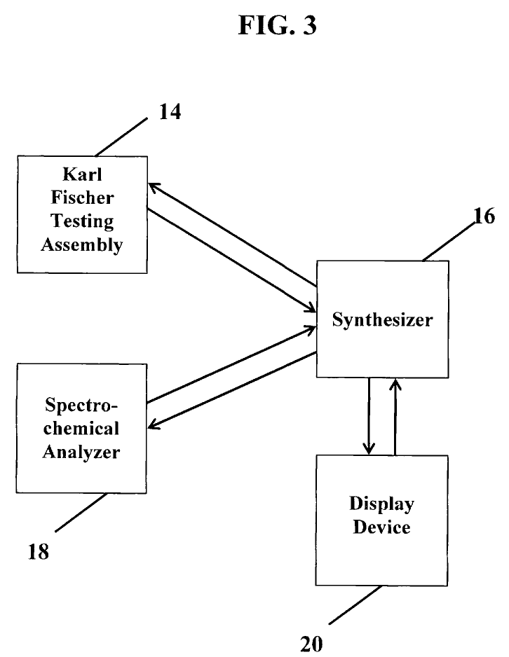

In one preferred embodiment of the invention, an apparatus is used to perform the method described above. With reference to FIG. 3, the apparatus includes a Karl Fischer testing assembly 14 in communication with a synthesizer 16. A spectrochemical analyzer 18 is also in communication with the synthesizer 16. During operation, a Karl Fischer test is performed on a first sample of an oil mixture at the Karl Fischer testing assembly 14. A Karl Fischer test result is communicated to the synthesizer 16. Also during operation, a spectrochemical analysis is performed on a second sample of the oil mixture at the spectrochemical analyzer 18, thereby producing composition data. The composition data is communicated to the synthesizer 16. After or substantially when the synthesizer 16 receives the Karl Fischer test result from the Karl Fischer testing assembly 14 and the composition data from the spectrochemical analyzer 18, the synthesizer 16 analyzes the Karl Fischer test result and the composition data to produce a water content measurement. The water content measurement better corresponds with the actual water concentration of the oil mixture because the masquerading effects of other chemical species have been accounted for and calculated into the measurement.

The Karl Fischer testing assembly 14 is broadly defined herein as any testing assembly known to those skilled in the art that can be used to perform a Karl Fischer test. The “first sample” as defined above includes only the portion of the oil mixture depleted in the chemical reaction to produce a Karl Fischer result. The “second sample” as defined above includes only the portion of the oil mixture depleted in the spectrochemical analyzer to produce composition data. In a preferred embodiment, the spectrochemical analyzer 18 includes a spectrometer, a spectrograph, or a spectroscope. In another preferred embodiment, the synthesizer 16 includes a computer, and the computer 16 is preferably in communication with a display device 20 for displaying the water content measurement from the computer 16 as shown in FIG. 3. In yet another embodiment, the synthesizer 16 includes a human who receives the Karl Fischer test result and the composition data via visual, auditory, or other human sensory function and produces a water content measurement based on the Karl Fischer test result and the composition data.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method for determining a concentration of water in an oil mixture composed of oil, water and at least one other chemical species, the method comprising the steps of:
a. using the Karl Fisher test method to determine a Karl Fisher test result corresponding to the concentration of water in the oil mixture;
b. determining a concentration of the at least one other chemical species in the oil mixture using spectrochemical analysis; and
c. calculating an accurate concentration measurement of water in the oil mixture based on the Karl Fisher test result and the concentration of the at least one other chemical species.

2. The method of claim 1 wherein the at least one other chemical species comprises magnesium sterate.

3. The method of claim 1 wherein the at least one other chemical species comprises ZDDP.

4. The method of claim 1 wherein using spectrochemical analysis includes the use of a spectrometer.

5. The method of claim 1 wherein using spectrochemical analysis includes the use of a spectroscope.

6. The method of claim 1 wherein using spectrochemical analysis includes the use of a spectrograph.

7. The method of claim 1 further comprising the step of performing a crackle test on the oil mixture to determine if water is present in the oil mixture.

8. An apparatus for determining the actual concentration of water in an oil mixture, the apparatus comprising:
a. a Karl Fisher testing assembly for performing the Karl Fisher test method on a first sample of the oil mixture to produce a Karl Fisher test result;
b. a spectrochemical analyzer for determining composition data corresponding to a chemical composition of the oil mixture using a second sample of the oil mixture; and
c. a synthesizer for receiving the Karl Fisher test result from the Karl Fisher testing assembly, receiving the composition data from the spectrochemical analyzer, and analyzing the Karl fisher test result and the composition data to produce a water content measurement that better corresponds to the actual concentration of water in the oil mixture than the Karl Fischer test result.

9. The apparatus of claim 8 wherein the spectrochemical analyzer is selected form the group consisting of a spectrometer, a spectroscope, and a spectrograph.
